(19) United States
(12) Patent Application Publication
(10) Pub. No.: US 2009/0287223 A1
(43) Pub. Date: Nov. 19, 2009

(54) REAL-TIME 3-D ULTRASOUND GUIDANCE OF SURGICAL ROBOTICS

(76) Inventors: Eric Pua, Durham, NC (US); Edward D. Light, Durham, NC (US); Daniel Von Allmen, Chapel Hill, NC (US); Stephen W. Smith, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/307,628

(22) PCT Filed: Jul. 11, 2007
(86) PCT No.: PCT/US07/15780

§ 371 Date: Jan. 6, 2009

Related U.S. Application Data
(60) Provisional application No. 60/819,625, filed on Jul. 11, 2006.

(51) Int. Cl. A61B 19/00; A61B 8/00; A61B 5/05

(52) U.S. Cl. 606/130; 600/443; 600/426

(57) ABSTRACT

Laparoscopic ultrasound has seen increased use as a surgical aide in general, gynecological, and urological procedures. The application of real-time three-dimensional (RT3D) ultrasound to these laparoscopic procedures may increase information available to the surgeon and serve as an additional intraoperative guidance tool. The integration of RT3D with recent advances in robotic surgery can also increase automation and ease of use. In one non-limiting exemplary implementation, a 1 cm diameter probe for RT3D has been used laparoscopically for in vivo imaging of a canine. The probe, which operates at 5 MHz, was used to image the spleen, liver, and gall bladder as well as to guide surgical instruments. Furthermore, the 3D measurement system of the volumetric scanner used with this probe was tested as a guidance mechanism for a robotic linear motion system in order to simulate the feasibility of RT3D/robotic surgery integration. Using images acquired with the 3D laparoscopic ultrasound device, coordinates were acquired by the scanner and used to direct a robotically controlled needle towards desired in vitro targets as well as targets in a post-mortem canine. The RMS error for these measurements was 1.34 mm using optical alignment and 0.76 mm using ultrasound alignment.

### FIELD

The technology herein relates to the use of real-time 3D ultrasound in a laparoscopic setting and in percutaneous procedures and as a direct guidance tool for robotic surgery.

### BACKGROUND AND SUMMARY

Robotic surgery technology has made recent gains as an accepted alternative to traditional instruments in cardiovascular, neurological, orthopedic, urological, and general surgery. With the da Vinci system (Intuitive Surgical, Inc., Sunnyvale, Calif.), a multi-camera endoscope is used for 3D visualization, increasing visibility and depth perception for the robot operator. The dual-lens endoscope links to two monitors, enabling 3D stereoscopic vision within the patient. The robotic arms also exhibit precise, dexterous control, eliminating tremor and improving ergonomics for the surgeon. For laparoscopic procedures, there have been published reports of using robotics in cases of splenectomy, adrenalectomy, cholecystectomy, and gastric bypass among others. In most cases, surgeons reported better visualization, increased instrument control, reduced operator fatigue, and an improved learning curve for those training to perform these procedures.

Also in recent years, the development of endoscopic transducer designs has enabled the application of B-scan laparoscopic ultrasound as a preoperative and intraoperative tool for assistance in surgical guidance and assessment. The primary advantage of laparoscopic ultrasound (LUS) is the ability to image beyond tissue boundaries. However, optical laparoscopes generally provide only views of the outer surface of organs, and laparoscopic graspers can generally only give a rudimentary feedback regarding tissue texture or underlying masses. The integration of LUS into the operating room provides visualization of most surrounding soft tissue structures, allowing access to information that might otherwise only be available in an open surgery setting. Additionally, the ability to place the transducer directly against an organ allows the use of higher frequency devices, which provide better resolution.

Laparoscopic ultrasound has been used effectively during minimally invasive surgeries and for cancer staging in the liver and in urological applications. For cancer staging, LUS is utilized for tumor detection, localization and border definition, and post-operative analysis. Combined with endoscopic ultrasound, it has been used for localization of gastric submucosal tumors targeted for resection. In addition to gastric and hepatic cancers, LUS has also been employed as an aid for treatment of pancreatic and adrenal tumors. Furthermore, there has been an increase in investigations using laparoscopic ultrasound in gynecological cases, such as the treatment of uterine myomas.

The application of real-time three-dimensional (RT3D) ultrasound imaging may increase the utility of laparoscopic ultrasound for these applications. While acquiring full volumes of information intraoperatively, real-time 3D ultrasound may provide improved visualization and possibly decrease procedure time and difficulty. The ability to visualize multiple planes through a volume in real-time without moving the transducer can improve determination of target geometry as well. Acquisition of volumetric data with RT3D is achieved through the use of one dimensional arrays combined with a motor or with two-dimensional transducer arrays and sector phased array scanning in both the azimuth and elevation directions. In this way, pyramidal volumes of data, as shown in FIG. 1, are acquired without the use post-acquisition reconstruction.

Real-time 3D ultrasound has been used in a variety of contexts. Transthoracic echocardiographic studies using RT3D have been effective for applications such as monitoring left ventricular function, detecting perfusion defects, and evaluating congenital abnormalities. More recently, catheter-based transducers with two-dimensional arrays have been developed for intracardiac echocardiography. These devices have been fabricated into 7F catheters with as many as 112 channels, successfully merging functional intracorporeal size with clinically-relevant resolution for RT3D. Advances from the design of intracardiac catheters have been the catalyst for the recent fabrication of endoscopic and laparoscopic 3D probes which have been employed for cardiac applications. These have been constructed with 504 active channels at operating frequencies ranging from 5 to 7 MHz. These latter devices are also well-suited for assisting in laparoscopic surgeries, serving as a preoperative tool as well as a means of intraoperative guidance.

An additional advantage that RT3D laparoscopic ultrasound provides over conventional 2D LUS is the ability to establish a true 3D coordinate system for measurement and guidance. Traditional ultrasound scanner systems are capable of two-dimensional measurements. With volumes of data acquired in real-time, RT3D scanners can provide a surgeon with three-dimensional structural orientation within a target organ using its measurement system, providing more information than was previously available. This could be particularly useful in conjunction with recent advancements in robotic surgeries. With new equipment such as the da Vinci robotic surgical system, the integration of RT3D and its measurements can help locate targets and steer the robot's arms into position, while avoiding regions that must not be damaged.

In one non-limiting exemplary implementation, a 1 cm diameter probe for RT3D has been used laparoscopically for in vivo imaging of a canine. The probe, which operates at 5 MHz, was used to image the spleen, liver, and gall bladder as well as to guide surgical instruments. Furthermore, the 3D measurement system of the volumetric scanner used with this probe was tested as a guidance mechanism for a robotic linear motion system in order to simulate the feasibility of RT3D/robotic surgery integration. Using images acquired with the 3D laparoscopic ultrasound device, coordinates were acquired by the scanner and used to direct a robotically controlled needle towards desired in vitro targets as well as targets in a post-mortem canine. The RMS error for these measurements was 1.34 mm using optical alignment and 0.76 mm using ultrasound alignment.

### BRIEF DESCRIPTION OF THE DRAWINGS

These and other features and advantages will be better and more completely understood by referring to the following detailed description of exemplary non-limiting illustrative embodiments in conjunction with the drawings of which:

FIG. 1 is a schematic of an exemplary illustrative non-limiting real-time 3D laparoscopic probe used in conjunction with a robotic device for surgical guidance;

FIG. 2 is a close-up of an exemplary illustrative non-limiting 3D laparoscopic probe (A) with a 4-directional bending sheath and 6.3 mm×6.3 mm aperture and a (b) 5 mm diameter Endopath surgical forceps;

FIGS. 3A, 3B, 3C are example images of a 12 mm hypoechoic lesion in a tissue-mimicking medium;

FIGS. 4A 4B, 4C, 4D, 4D are example images of simultaneous optical laparoscopic views of the liver and gall bladder;

FIGS. 5A, 5B, 5C and 5D are example images of simultaneous optical laparoscope views;

FIGS. 6A, 6B are example images of stereoscopic imaging with real-time 3D ultrasound;

FIGS. 7A, 7B, 7C show exemplary illustrative non-limiting ultrasound guidance of a robotically controlled 1.33 mm diameter needle using B-scan image measurements;

FIGS. 8A, 8B and 8C show 3D exemplary illustrative non-limiting ultrasound guidance of a robotically controlled 1.3 mm needle using 3D ultrasound measurements;

FIGS. 9A, 9B show exemplary illustrative non-limiting integrated 3D ultrasound guidance and robotics for a hypo-echoic lesion in a tissue-mimicking medium;

FIGS. 10, 10A, 10B, 10C, 10D, 10E, 10F show split-screen video captures of a 15 cm needle puncturing the gall bladder of a canine cadaver; and

FIG. 11 shows an exemplary illustrative non-limiting alternate implementation of 3D ultrasound guidance of the surgical robot.

### DETAILED DESCRIPTION

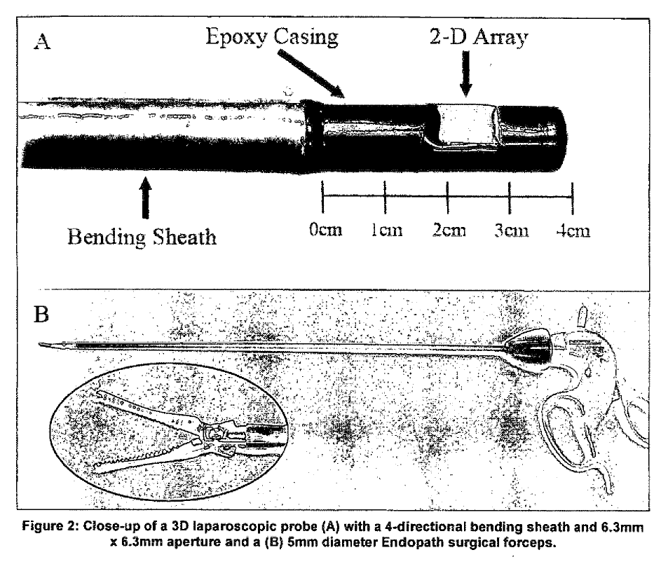

The technology herein relates to the use of real-time 3D ultrasound in a laparoscopic setting and as a direct guidance tool for robotic surgery. A steerable RT3D probe (FIG. 2A) can be modified for use as a laparoscope and utilized for in vivo imaging of a canine model. During a minimally invasive procedure, this probe can be used to produce volumetric scans of the liver, spleen, gall bladder, and introduced hypoechoic targets.

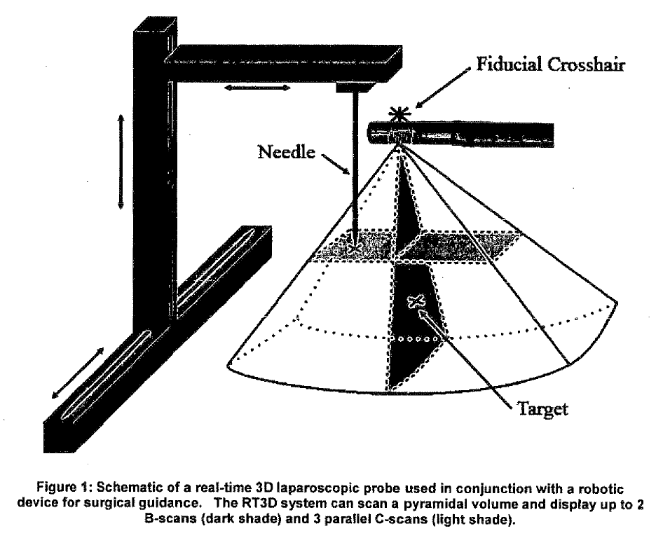

In addition, the probe can be used in vitro in conjunction with an exemplary illustrative non-limiting RT3D measurement system and a robotic linear motion system to demonstrate use of RT3D for semi-automated guidance of a surgical robot. A simplified schematic of the two systems working in concert is shown in FIG. 1. The combination of RT3D and robotics for laparoscopic surgery may improve procedure accuracy and decrease operation time and difficulty. Integration of the two systems can also increase automation in cases such as biopsies and allow for the establishment of regions where the robotic instruments must not operate.

FIG. 1 shows an exemplary illustrative non-limiting Scanner System and 3D Laparoscopic Probe. A real-time 3D ultrasound scanner system such as manufactured by Volumetrics Medical Imaging, Durham, N.C. can be used. The exemplary illustrative non-limiting implementation employs up to 512 transmitters and 256 receivers with 16:1 receive mode parallel processing. The exemplary illustrative non-limiting system is capable of acquiring 4100 B-mode image lines at a rate of up to 30 volumes per second with scan angles from 60 to 120 degrees. This acquisition produces, for example, a pyramidal volume equivalent to 64 sector scans of 64 lines each, stacked in the elevation dimension. The exemplary system's display scheme permits the simultaneous visualization of 2 standard orthogonal B-scans as well as up to three C-scan planes parallel to the face of the transducer array. The B-scans and C-scans can be tilted at any angle while the angle and depth of the C-scans can be changed in real-time. Integration and spatial filtering of the data encompassed by two C-scan planes provides a real-time volume-rendered image. Of course, other alternative scanner system implementations could be used instead.

In one exemplary illustrative non-limiting implementation, a real-time scan converter transforms echo data from the 3D spherical (r, θ, φ) geometry of the pyramidal scan to the rectangular (x, y, z) geometry of a television or other display. The xyz coordinates provided by the measuring program for distance, area, and volume calculations can be derived from scan converter viewport tables that generate the image slices for the display. These viewport tables in turn are assembled from a 3D cubic decimation/interpolation system on the scan conversion hardware, which accept the depth, azimuth angle, and elevation angle from the received echo data and convert them to rectangular coordinates. The original documented measurement error of an exemplary illustrative non-limiting system is shown in Table 1. These values reflect the average length error in measurements made in the designated scan type over the given range of depth. Variability over this target range is generally provided by the manufacturer of a particular system.

TABLE 1
Documented Measurement Error of Volumetrics System

| Scan Type | Target Depths | % Error |
|---|---|---|
| B-Scan | 3-12 cm | 2.31 |
| C-Scan | 3-12 cm | 5.5 |

One exemplary illustrative non-limiting implementation employs a transducer comprising a 504 channel matrix array probe originally designed for transesophageal echocardiography, as shown in FIG. 2A. A 4-directional bending sheath is incorporated into the tip of the probe. This steering function also provides quick orientation adjustment in any direction. One exemplary illustrative non-limiting 3D TEE probe operates at 5 MHz using a 6.3 mm×6.3 mm aperture and has an outer diameter at the probe tip of 1 cm.

### RT3D Measurement for Robotic Guidance

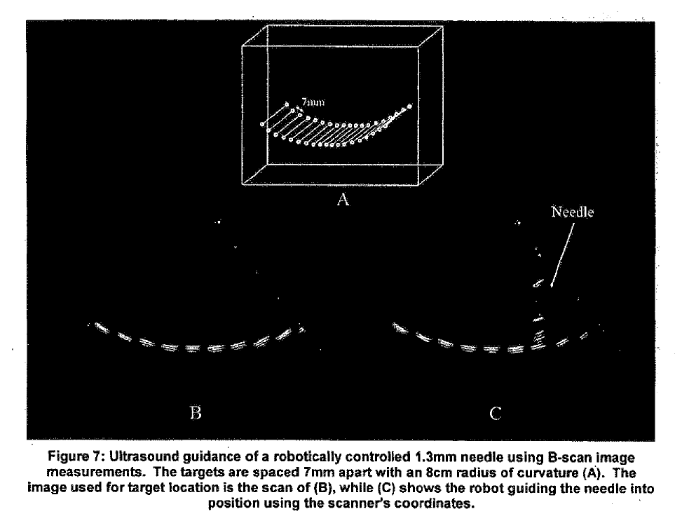

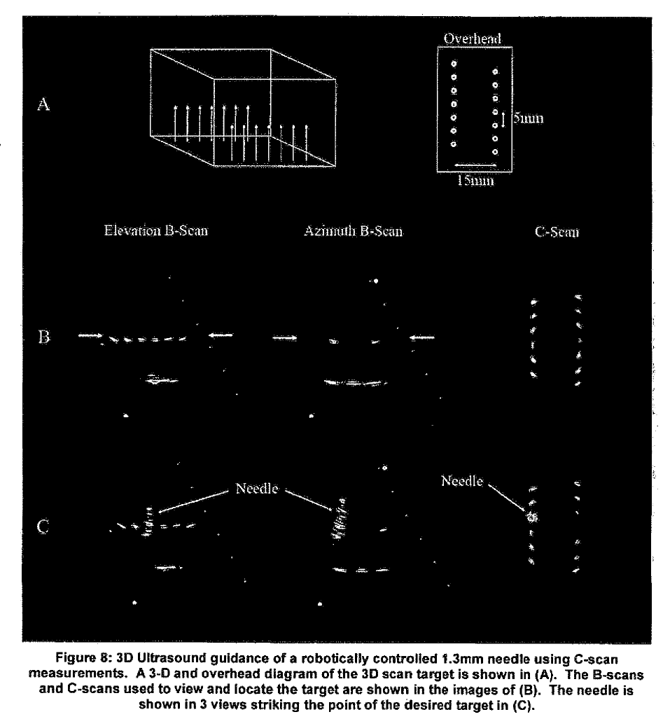

An example illustrative non-limiting robot of the FIG. 1 system comprises a Gantry III Cartesian Robot Linear Motion System manufactured by Techno-Isel (Techno, Inc., New Hyde Park, N.Y.). A simplified representation of this device is shown in FIG. 1. The exemplary illustrative non-limiting implementation employs a Model H26T55-MAC200SD automated controller which accepts input commands and 3-dimensional coordinates from a connected PC. The XY stage (model HL32SBM201201505) is a stepper motor design providing 340 mm×290 mm of travel on a 600 mm×500 mm stage. The Z-axis slide (model HL31SBM23051005) provides 125 mm of vertical clearance and allows 80 mm of travel in the z-dimension. An accuracy profile of the illustrative measurement system in coordination with a robotic device can be acquired. Three different measurement targets may be used for accuracy measurements. For example, a B-scan target may consist of 19 wire targets in a water tank spaced 7 mm apart with an 8 cm radius of curvature (FIG. 7A). A 3D scan target can be constructed of 2 rows of 7 vertically-oriented wire targets spaced 5 mm apart (FIG. 8A), with the two rows separated by 15 mm. The third phantom can be a 3 cm diameter hypoechoic lesion inside a tissue-mimicking slurry.

For these measurements, the aforementioned 3D laparoscopic probe, connected to the scanner, may be flexed at the bending sheath ninety degrees in the elevation plane in order to face downwards into a water tank or tissue-mimicking medium, located on the XY stage of the Cartesian robot. A fiducial crosshair, illustrated in FIG. 1, can be etched into the back of the 3D probe for optical alignment of a robotically-guided 1.2 mm diameter needle with the center of the transducer face. Once the TE probe transducer face is aligned so as to provide a view of the desired measurement targets on the scanner, the needle may be centered on the back of the transducer using the robot controller. The scanner may then be used to image the target. Once frozen, target coordinates can be taken using the scanner measurement system. With the robot's frame of reference zeroed on the transducer's fiducial spot, these coordinates may be input into the robot controller, allowing for the 1 cm thickness of the probe. Once the robot has positioned the needle according to the coordinates predicted by the 3D image, the tip may be repositioned via the robot's stepping function in 0.1 mm increments in three dimensions until it makes contact with the target. Visual confirmation of contact may be used to determine whether repositioning is complete. The adjusted coordinates from the robot controller may be recorded in order to calculate RMS error. For example, a series of 10 measurements can be taken for the B-scan target phantom, and 16 data points may be collected for the 3D scan targets. This data may be collected over several trials (the phantom and transducer setup can be dismantled at the end of each experiment). The use of optical alignment with a fiducial spot is also applicable to any 3D ultrasound transducer including transducers located on the surface of the body for percutaneous minimally invasive procedures.

An additional 12 measurements may be taken in the 3D scanning mode for ultrasound alignment without centering the needle on the fiducial crosshair. In this setup, the transducer may be flexed in the opposite direction and placed at the bottom of the water tank, facing upwards. The guided needle can be lowered until it is visible at an arbitrary location in one of the B-scan or C-Scan displays. The other B-scan slice may be selected to show one of the 3D scan targets. Coordinates for the tip of the needle (x, y, z) and the desired target (x′, y′, z′) may be acquired using each B-scan or a C-scan plane, and the differences (Δx=x−x′, Δy=y−y′, αz=z−z′) can be calculated. The needle may then be moved by αx, αy, αz relative to its original location in order to make contact with the 3D scan target. RMS Error measurements may be recorded using 0.1 mm increments, as stated before.

In an additional experiment, the optical alignment method may be used for guiding a needle towards a designated target on the organ boundaries in a post-mortem canine. For this study, a fresh canine cadaver can be placed on the XY stage of the robot, and an approximately e.g., 30 cm long incision can be made to open the abdomen, starting at the base of the sternum. The RT3D probe can be flexed into the downward facing position, and the array face may be placed in contact with the liver and gall bladder. Optical alignment can be used for centering the tip of a 1.2 mm diameter, 15 cm long needle on the fiducial crosshair. The scanner can be used to determine the distance for the needle to travel in order to puncture the distal boundary of the gall bladder at a desired location. Visualization of the needle movement may be recorded for example with a CCD camera simultaneously with the real-time 3D scans using a video screen-splitting device.

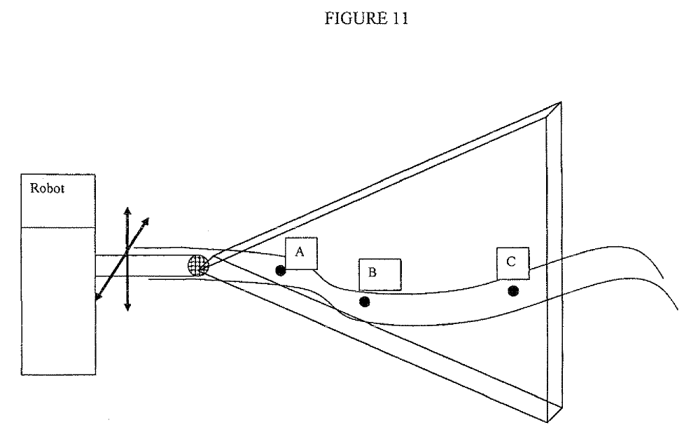

FIG. 11 shows an alternate implementation of 3D ultrasound guidance of the surgical robot which may be useful for interventional cardiology or radiology. The figure above uses a 3D ultrasound catheter or endoscope with a forward scanning matrix array and four directional mechanical steering shown by the double arrow incorporated into a robot arm. The 3D ultrasound scanner with the catheter/endoscope measures the location of anatomical landmarks denoted by A,B,C within a convoluted structure such as a blood vessel or bowel using the 3D ultrasound images as described above. Knowing the location of the landmarks the robot can plot a course down the vessel or bowel by advancing the catheter/endoscope to a desired location. The robot arm can advance to one landmark at a time and recalibrate its position or can plot an overall path using a technique such as cubic spline.

### Exemplary Illustrative Non-Limiting Results and Images

### Example

### Animal Model and 3D Laparoscopic Study

The Institutional Animal Care and Use Committee approved the use of a canine model for the acquisition of in vivo 3D images, conforming to the Research Animal Use Guidelines of the American Heart Association. Ketamine hydrochloride 10-15 mg/kg IM was used to sedate the dog. An IV of 0.9% sodium chloride was established in the peripheral vein and maintained at 5 mL/kg/min. Anesthesia was induced via nasal inhalation of isoflurane gas 1-5%. An endotracheal tube for artificial respiration was inserted after oral intubation with the dog placed on its back on a water-heated thermal pad. A femoral arterial line was placed on the left side via a percutaneous puncture. Electrolyte and respirator adjustments were made based on serial electrolyte and arterial blood gas measurements. Blood pressure, electrocardiogram, and temperature were continuously monitored throughout the procedure.

After the animal preparations were complete, the dog's abdominal cavity was insufflated with carbon dioxide gas. Four surgical trocar ports were introduced into this cavity. One port was designated for an optical laparoscope while two others were used primarily for surgical forceps and introducing imaging targets. The 3D laparoscopic ultrasound probe was introduced into the fourth port with its bending sheath flexed to 90 degrees in order to facilitate contact with the canine's organs. The probe was guided to the desired locations using the optical laparoscope. Once all instruments were in place, images of the spleen, liver, and gall bladder were acquired before and after introduction of forceps. In addition, an XXL™ balloon dilatation catheter (Boston Scientific, Watertown, Mass.) was introduced into the liver and the spleen to provide a hypoechoic imaging target for the 3D probe to locate. The catheter is a 5.8 Fr device with an inflated balloon size of 12 mm by 2 cm. All imaging and surgical procedures were monitored via the optical laparoscope.

Real-time images of in vivo canine anatomy and robotic surgical targeting were acquired with the Model V360 and Model 1 Volumetrics scanners interfaced with the described 3D laparoscopic probe. These images include user-selected 60 degree and 90 degree B-scans, C-scans, and 3D volume-rendered scans. The intersections of multiple B-scan planes are indicated by blunt arrowheads at the base of elevation and azimuth scans, while larger arrows to the sides indicate the planes used for each C-scan or volume-rendered image. The depth scale of each scan is shown by the white dots along the sides of each B-scan, each dot indicating 1 centimeter. The scale of the 3D rendered images does not directly correspond with that of the corresponding B-scans.

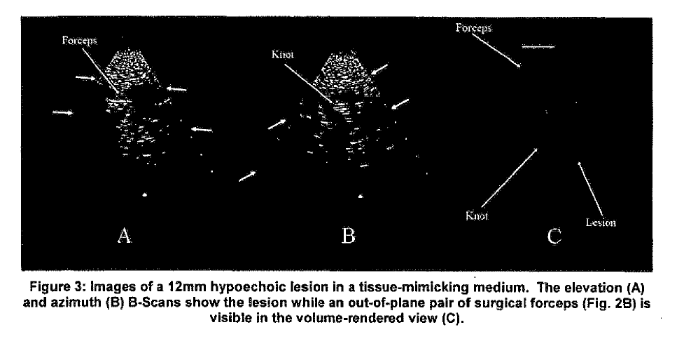

In FIG. 3, the in vitro image quality and volume rendering capabilities of the 3D laparoscopic probe are shown. The image was taken from an 8 cm deep, 60° 3D scan of a tissue-mimicking slurry with a 12 mm hypoechoic lesion (water balloon) suspended in the medium. The elevation B-scan (FIG. 3A) shows the full diameter of the lesion. Barely visible in this view is the stem of a 5 mm Endopath surgical forceps instrument (Ethicon Endo-Surgery) (FIG. 2B). The azimuth B-scan (FIG. 3B) shows a portion of the lesion and the knot from which it is anchored. The knot of the target produces shadowing throughout the rest of the scan. The forceps are only clearly visible in the volume rendered view (FIG. 3C), which has been acquired using the data between the planes indicated by the arrows. In this image, the open forceps are rendered in the foreground with the lesion and its point of attachment in the background.

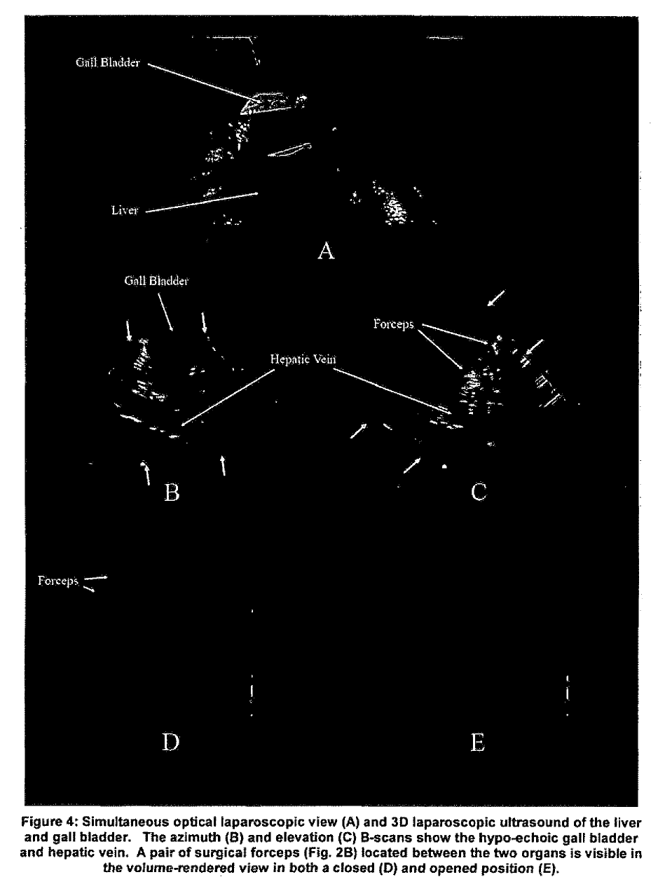

FIG. 4 shows a 4 cm deep, 90 degree scan of the gall bladder. In FIG. 4A, the transducer face is placed against the gall bladder with liver tissue surrounding it. A short axis (FIG. 4B) and long axis (FIG. 4C) view of the gall bladder are both visible in the displayed B-scans. Also visible in these B-scans are a long axis (FIG. 4B) and short axis (FIG. 4C) view of the hepatic vein, approximately 5 mm in diameter. Not shown in the optical view, surgical forceps (FIG. 2B) are present between the surfaces of the gall bladder and the liver of the canine. The jaws of the forceps can be seen both partially closed and opened in the volume-rendered images (FIG. 4D-E). The renderings were acquired using the ultrasound data between the C-scan planes indicated by the arrows. Close inspection of the B-scans shows cross-sectional views of the two points of the forceps in FIG. 4C. The views of the forceps in FIGS. 3-4 demonstrate the value of real-time 3D rendering over the selected slices from a 3D scan.

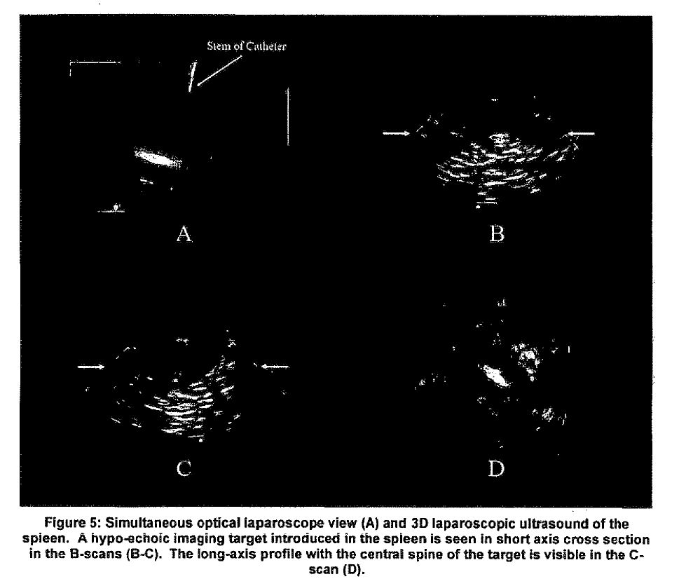

For imaging the spleen, a balloon dilatation catheter was inserted to serve as a hypoechoic structure. The transducer placement over the spleen can be seen in FIG. 5A, with the stem of the balloon catheter located approximately 2 cm superior to the probe. Orthogonal short axis, cross-sectional views of the inflated balloon are shown in the B-scan slices (FIG. 5B-C) using a 4 cm deep, 90 degree scan. The profile of the hypoechoic target is shown in the C-scan (FIG. 5D). The bright target at the center of the balloon is the central spine of the catheter device from which the outer layer inflates.

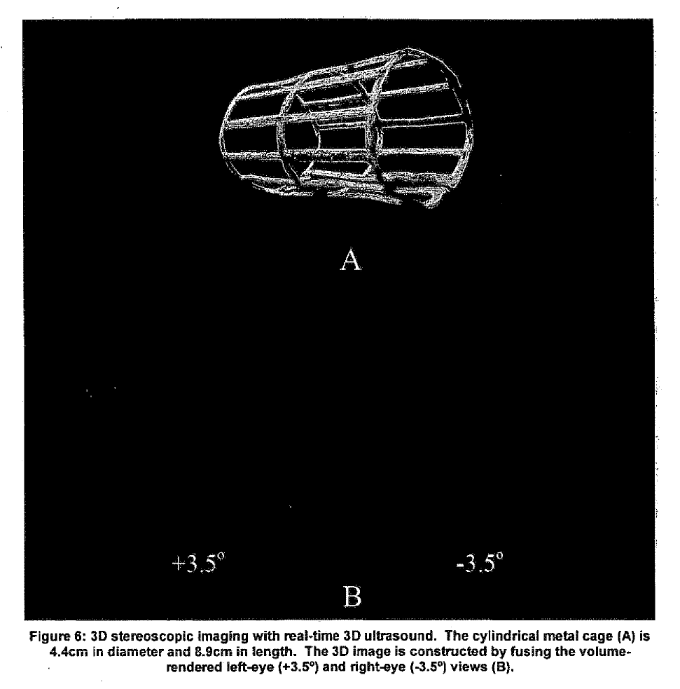

FIG. 6 shows a real-time stereoscopic display for the 3D scanner. The imaging target shown in FIG. 6A is a cylindrical metal cage 4.4 cm in diameter and 8.9 cm in length. A 65° 3D scan was used to image down the length of the target, and volume rendering planes were set to display the foremost half of the cylinder. Once the scan was acquired, separate left-eye (+3.5°) and right-eye (−3.5°) views of the volume-rendered target are shown simultaneously on the screen, as shown in FIG. 6B. These two views can be fused by the observer as a stereoscopic pair, allowing for a 3D visualization of the target analogous to the dual-camera system used in the da Vinci robot system.

### Exemplary Illustrative Non-Limiting Robotic Guidance Accuracy

FIG. 7 shows the B-scan phantom with a 6 cm deep, 90 degree single B-scan. In FIG. 7B, 9 wires are clearly visible in cross-section. In FIG. 7C, the 3^{rd} wire from the right is in contact with the robot-controlled needle probe. For the single B-scan mode of the scanner, the RMS guidance error from measurements was found to be 0.86 mm±0.51 mm using optical alignment. Similarly, in FIG. 8B, orthogonal B-scans and a C-scan of the 3D phantom are shown before the needle has been positioned. These images were attained with a 6 cm deep, 60 degree 3D scan. An entire row is visible in the elevation B-scan while a pair of targets from the two rows is shown in the azimuth B-scan. The C-scan shows the profile of the 3D phantom with all the target tips clearly visible. In FIG. 8C, the Cartesian robot has positioned the needle to come into contact with a target in the left column, as visible in all 3 image planes of the scan. The mean RMS error for these 3D scan measurements was found to be 1.34 mm±0.68 mm using optical alignment.

A third set of measurements was taken without the use of the optical fiducial mark, using only ultrasound alignment. These yielded a mean RMS error of 0.76 mm±0.45 mm.

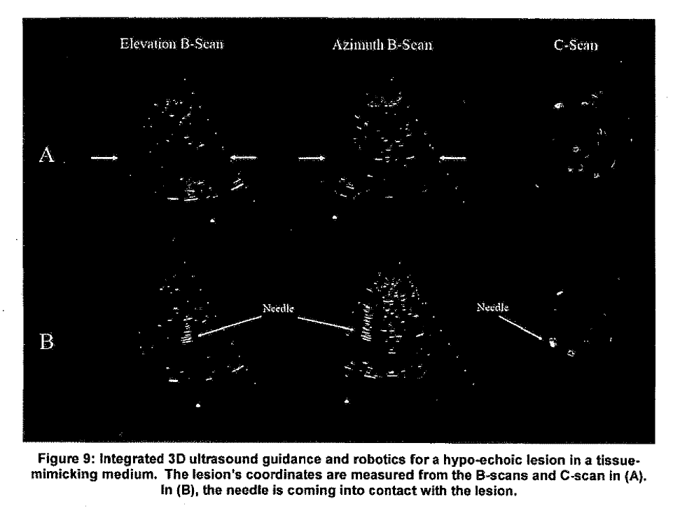

For the tissue-mimicking phantom, we performed several trials at making contact with the hypoechoic lesion using both C-scan and B-scan coordinates. In FIG. 9A, the needle has not yet been positioned with the robot, and the lesion is clearly visible in both B-scans and the accompanying C-scan. In FIG. 9B, it is evident that the needle has come into contact with the target. The needle tip appears to be deforming the lesion slightly, as it is visible within the diameter of the target in both B-scans and in the C-scan plane. Error measurements of the needle strike point compared to the measurement point on the scanner were not taken due to the optical opacity of the graphite slurry containing the lesion; however, scan plane markers were used to identify the desired position of needle placement. These markers give an approximation of the measurement error for the experiment.

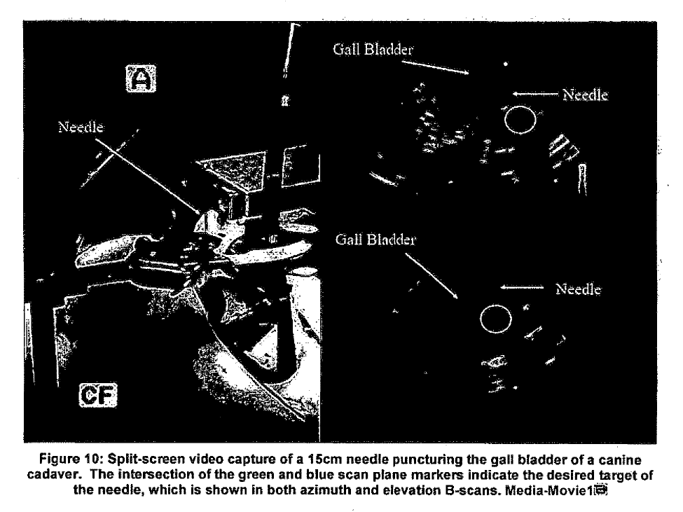

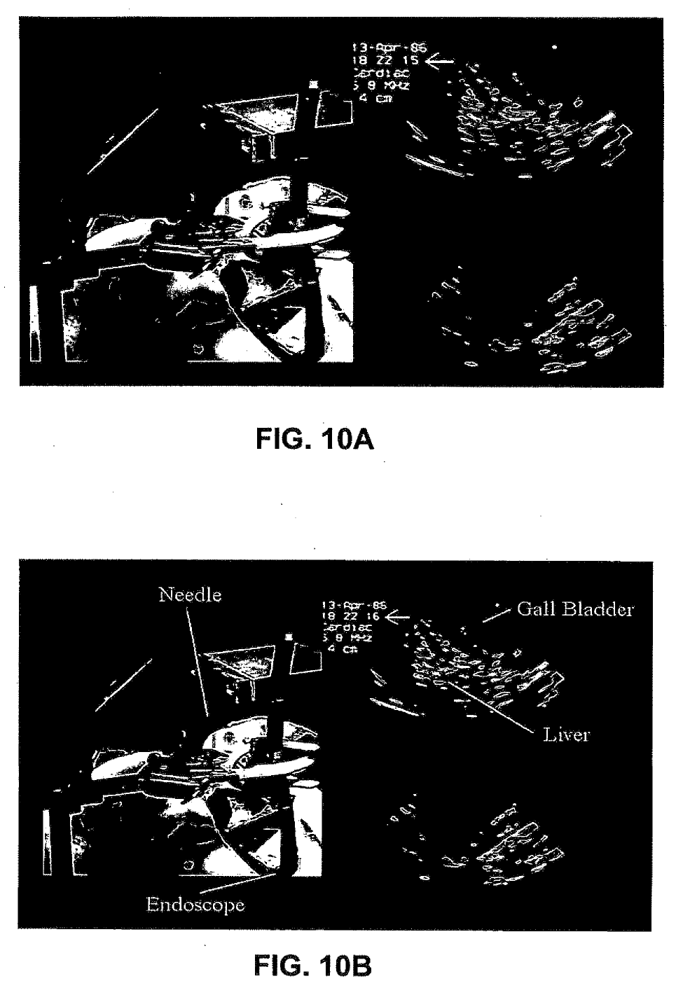

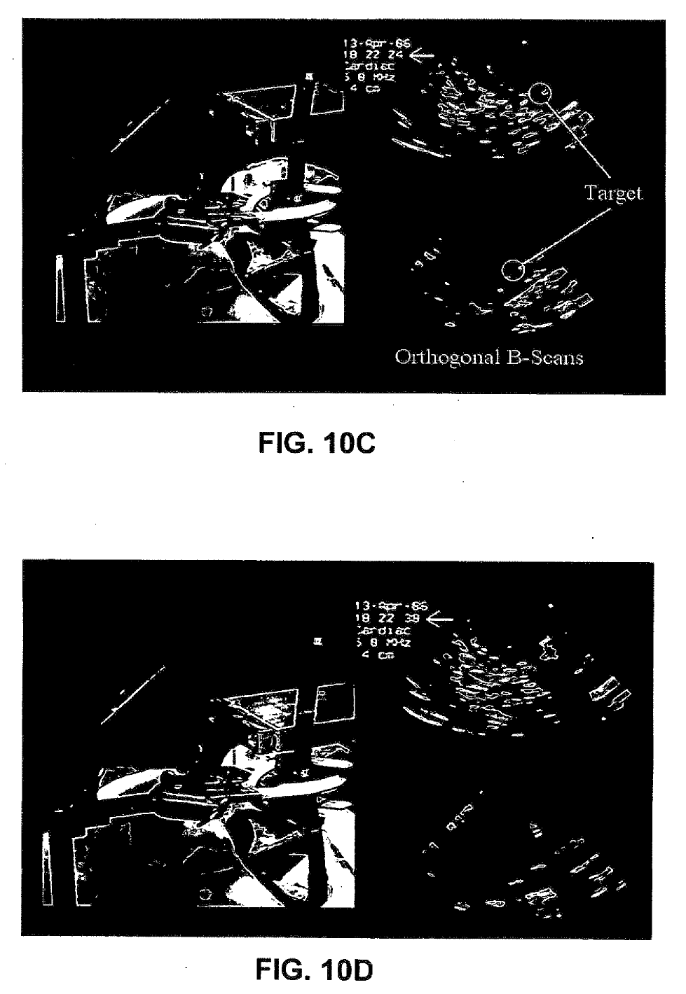

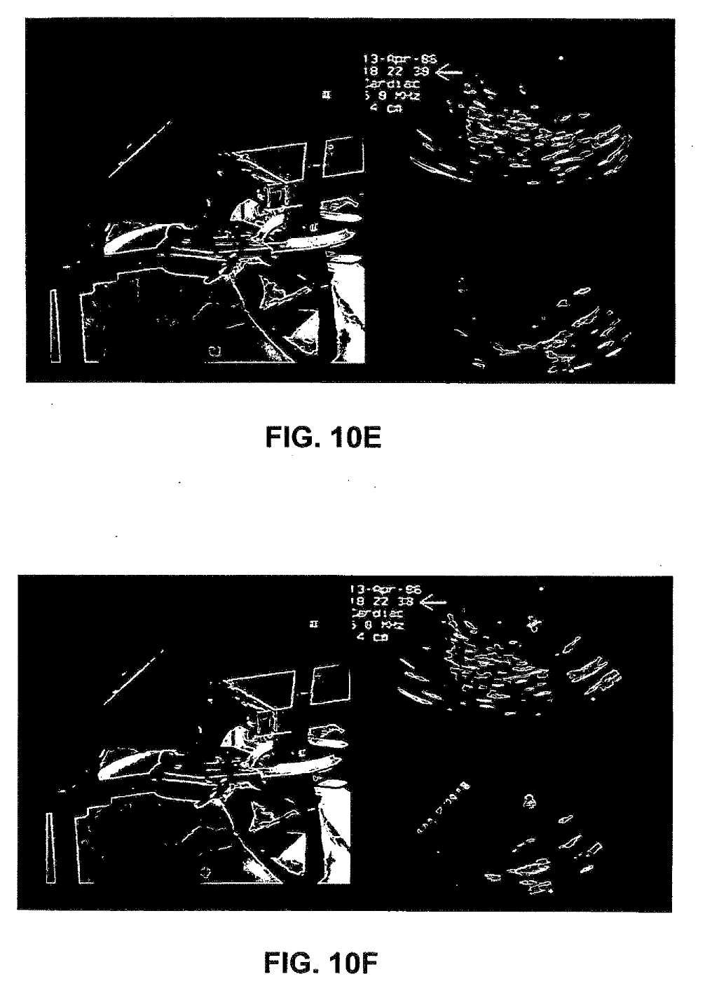

In a cadaver experiment, the coordination of the robotic motion system and 3D ultrasound measurements was employed to puncture a desired position on the distal wall of the gall bladder. FIG. 10 illustrates the procedure. First, coordinates were acquired at the desired location in the gall bladder, indicated by the white circles in the movie. These were monitored using the green and blue scan plane markers of the azimuth and elevation B-scans. The needle can be seen in the left view as it is lowered into the cadaver's abdomen. Meanwhile, in the right view, it is clearly reaching the designated target in both B-scans. There is a small error in the azimuth B-scan which appears to be on the order of 1-2 mm.

Using a 3D laparoscopic ultrasound probe, images of in vivo canine abdominal anatomy have been acquired. These scans (FIGS. 4-5) show the image quality indicative of current prototype endoscopic probes designed for real-time 3D ultrasound. From these pictures, it appears that such devices are well-suited for assisting in laparoscopic surgeries. In FIG. 4, volume rendered views provide visualization of surgical instruments that were not immediately noticeable in standard B-scans. Similarly, in FIG. 5, the combination of standard B-Scans with parallel C-scan views enables better spatial familiarity with the shape and size of the angioplasty balloon introduced into the spleen. In this set of images, the width, length, and interior structure of the target are all apparent simultaneously from the three displayed slices. The ability to view the acquired volumetric data stereoscopically can further enhance three-dimensional visualization of surgical instruments and the target region. These factors are encouraging for the application of RT3D to the laparoscopic surgery setting.

Some current limitations with this exemplary illustrative non-limiting technology are size, maneuverability, and the need for higher frequency operation. The articulation of the bending sheath is useful for maneuvering the side-scanning RT3D probe into position, particularly for the in vivo imaging. However, forward-looking 2D array devices may be better suited for these situations if a steering mechanism were incorporated. Also, one would ideally like to have a higher level of resolution close to the transducer face since most targets will be within the first few centimeters for a laparoscopic procedure. The image quality in this region can be improved with the use of a higher frequency, broader bandwidth probe, which could enable for the addition of multi-frequency operation.

The error when using the FIG. 1 exemplary illustrative non-limiting scanner 3D coordinates to guide a robotic linear motion system to a specified target is less than 2 mm. A possible reason for the discrepancy in errors between the measurement methods is the elimination of user error in the case of ultrasound alignment. In the case of optical alignment, centering of the needle on the fiducial crosshair is dependent on user subjectivity. The exemplary illustrative non-limiting system shown in FIG. 1 is capable of 3 degrees of freedom; so, further tests with more sophisticated robotic equipment may be useful to prove efficacy and accuracy. However, the ability to integrate the RT3D system with robot surgical units has much potential. The last set of measurements using only ultrasound metrics is even more encouraging, since optical alignment of the robotically-guided device and the imaging probe is not necessary. With a margin of error of approximately 1 mm, this means that catheters and endoscopes could image the target organ from outside the surgical field, thus improving flexibility for surgical procedures.

Additional methods for defining the positions of the surgical tools in the ultrasound scan can be used including magnetic sensors or electrostatic sensors or optical encoders. Alternatively establish local GPS system in room or on patient to measure relative position of transducer and interventional device. Local GPS system may be 6 dimensional magnetic locator such as Biosense Webster Carto system or alternative may be electrical sensor such as Medtronic Localisa or may be acoustic sensors. Alternatively measure outline coordinates of entire target; and using programmable milling machine type device, computer performs entire surgical or interventional procedure with minimal human input.

The combination of RT3D with robotic surgery systems could prove valuable when staging percutaneous or laparoscopic biopsies or for other surgeries when defining regions of the anatomy that the robot's instruments must automatically avoid. Current efforts may be focused on improved integration and on implementation for in vivo animal studies.

While the technology herein has been described in connection with exemplary illustrative non-limiting implementations, the invention is not to be limited by the disclosure. The invention is intended to be defined by the claims and to cover all corresponding and equivalent arrangements whether or not specifically disclosed herein.

1. A robotic system for use in medical procedures, comprising:
a real-time 3D ultrasonic probe;
a real-time 3D scanner coupled to said probe, said scanner scanning a volume and generating an output; and
a robot coupled to the real-time 3D scanner, said robot automatically performing at least one aspect of a medical procedure at least in part in response to said scanner output.

2. The system of claim 1 wherein said probe comprises an articulatable bending sheath.

3. The system of claim 1 wherein said robot includes a robotically-guided needle, and said probe has at least one fiducial mark for optical alignment with said robotically-guided needle.

4. The system of claim 1 wherein said probe has at least one fiducial mark.

5. The system of claim 1 wherein said robotic system is used for surgical procedures.

6. The system of claim 1 wherein said wherein said robotic system is used for laparoscopic procedures.
